(19) United States
(12) Patent Application Publication
(10) Pub. No.: US 2023/0285183 A1
(43) Pub. Date: Sep. 14, 2023

(54) HELMET

(71) Applicant: MALACHI INDIA YAHTUES, BRIDGEWATER, MA (US)

(72) Inventor: MALACHI INDIA YAHTUES, BRIDGEWATER, MA (US)

(21) Appl. No.: 17/803,151

(22) Filed: Mar. 8, 2022

(51) Int. Cl. A61F 6/04

(52) U.S. Cl. CPC A61F 6/04; A61F 2006/047

(57) ABSTRACT

An apparatus termed Helmet, designed to be applied to a restricted portion of the male penis, and secured from movement and possible STI's through said secured area, by a manufactured adhesive-strip.

Said helmet's design and application allows for greater sexual sensation and stimulation of the remaining exposed male penis during the interim of sexual intercourse. While providing a level of protection from veneral diseases and unwanted pregnancies.

### FIELD OF THE INVENTION

This invention relates to an improved prophylactic apparatus, designed to assist in greater sexual stimulation and sensation during sexual intercourse while said elements of it's design provide assurance against Sexual Transmitted Diseases, and contraception.

### BACKGROUND OF INVENTION

The prophylactic market is not well stock according to opinion. Basically, there's not enough options. STD's and STI's still burden our world. One of the main reason's for such a burden, is peoples refusal to practice safe-sex. With this said, most people use the excuse or reason for failing to employ prophylactic's in general. For the purposes of this conversation; I will focus on the differences between the Helmet and other Male and Female Condoms.

The female condom known as a Diaphragm, presents the common fustration of needing to be preparatorily placed within the vagina prior to intercourse. Whereas, the male condom requires the male penis to be fully erect in order to be applied properly. This interlude is known to fustrate users because of the complications of improper application, tear's in the condom prior to, or during intercourse and inaccurate size differential between the penis and said condom.

Because each prophylactic item herein has it's advantages and disadvantages. For the purposes of this thesis; I will focus on the disadvantages.

The female condom's common fustration amongst the female population is it's burdensome application and the lack of full sexual sinsation shared by both men and women. Take for example it's application. Diaphragms come in many different forms of rubbery material's. Nonetheless, the diaphragm is shaped like a sleeve on a shirt with a sealed or capped-off end, and the opposite end has an opening for penile penetration. The female applicant takes the capped-off end of said sleeve and insert's in in her vagina until it is behind the pubic-bone; leaving the open-end protruding outside the vagina entrance. Such a burdensome task often enspiring females to forgo the usage of said diaphragm (which is typically used for protection of STI's and Birth-control), for risque' unprotected sex.

Now the male condom also known as a rubber, is a tube-shaped prophylatic rubbery material. Which requires as previously stated, that said male penis must be fully erected in order to be applied correctly. Men have often complained about this requirement being an interruption of a passion-filled moment. It may seem as if such a trivial inconvenience is not grave enough of an issue to forgo total abstinence of protective measures. However, when you couple this fustration along with the lack of sexual sinsation experienced with wearing a condom; a vast majority of men choose to obstain from usage of said condom.

The concept of this claim supporting the invention termed Helmet; was birth out of those fustrations previously mentioned. The helmet is aimed at eliminating all these relevent problems. First, the helmet is easy to apply; just flip up the belly-of-lip exposing the adhesive-strip, remove the protective wax-coated-pealable-paper and apply said helmet to the very tip of the penis-head termed crown and the adhesive-strip will adhere to the designated area. Allowing for approximately-90%-to-95% full sensation of the male penis, while providing a level of prophylatic protection; and, said helmet can be applied before or during sexual engagement with minimal effort; on an erect or flaccid penis.

Because said helmet uses about 98%-to-99% percent less material than the male and female condoms. Said helmet is more environmentally friendly.

### BRIEF SUMMARY OF THE INVENTION

The Helmet is an applicator to the male penis, designed to allow for the maximum amount of sexual sensation and stimulation of the penis during sexual intercourse; while helping to stop the spread of Sexual Transmitted Diseases and Infections (STD's or STI's), along with unwanted pregnancies.

The Helmet's conception is a direct response to men and women who refuse to employ prophylactics during sexual intercourse because of loss of sensation or inconvenience associated with it's application. The helmet seeks to remove those negative opinions; by making it'self available as an option to forgoing any or all life saving prophylactics.

### BRIEF DESCRIPTION OF THE DRAWINGS

The accompanying drawings further describe the invention:

FIG. 1, Entire image in FIG. 1, is said helmet; and element 1 is said reservior and element 2 is said lip.

FIG. 2, Element 3 is said tank.

FIG. 3, Element 6 is termed adhesive-strip, and element 5 is said belly-of-lip.

FIG. 4, Element 4 is the entrance-of-the-helmet; and element 3 is a toppled view of said tank.

FIG. 5, Shows a positional view of element 4, said entrance-of-the-helmet.

FIG. 6, Shows the lining of the interior wall of said belly-of-lip.

FIG. 7, Shows a positional view of said element 6 said adhesive-strip attached to the total circumference of said belly-of-lip.

FIG. 8, Shows element 7, said wax-coated-pealable-paper attached to the total circumference of said adhesive-strip.

FIG. 9, Is a diagram of the entire male genitalia divided in sections to assist in the helmet's application. The corresponding sections are identified as such; section A is the entire penis head; section B is the area termed crown; section C is the urethra-tube; section D entire length of male penis or penis; section E is scrotum

FIG. 10, Is a demostration of how the helmet is applied and all the correspond-elements associated with it's application.

FIG. 11, Shows a side view of said helmet applied properly to the penis-head.

FIG. 12, Shows a demonstration of said helmet applied properly to a flaccid penis head.

FIG. 13, Shows a demonstration of said helmet applied properly to an erect penis-head.

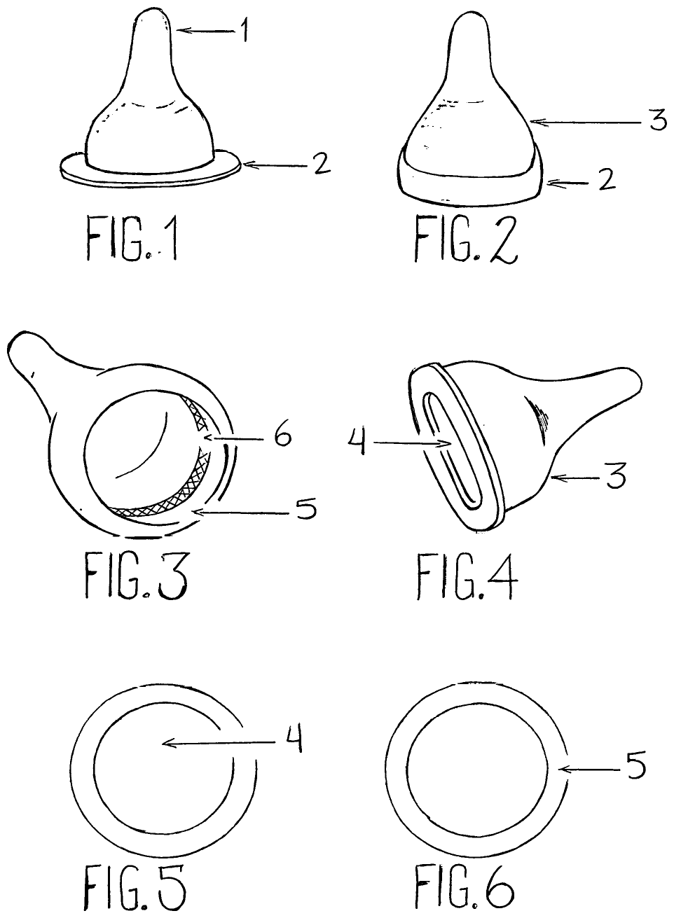

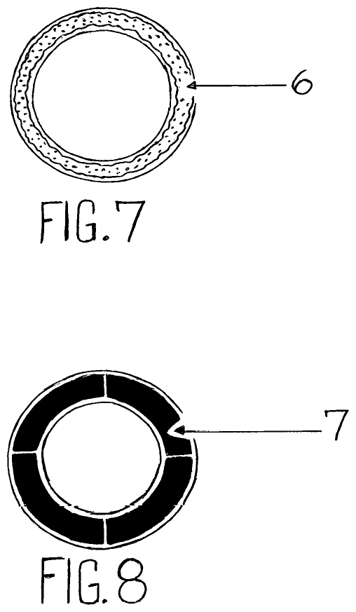

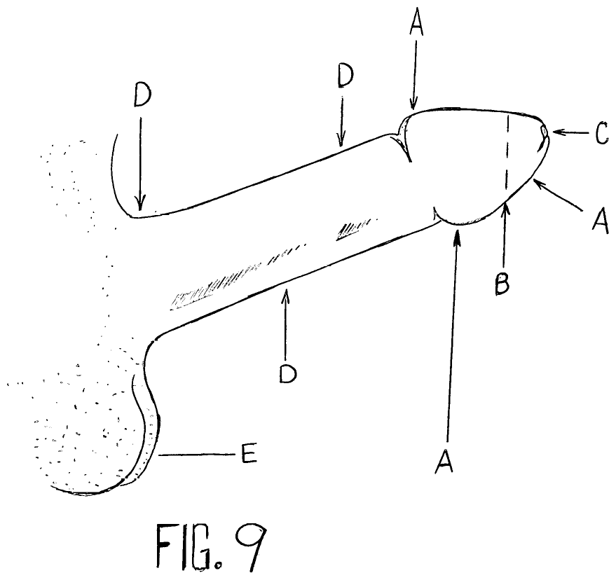

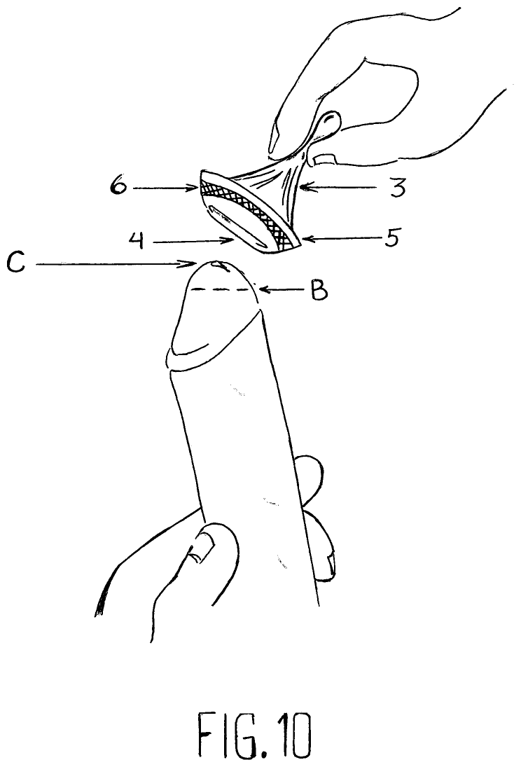

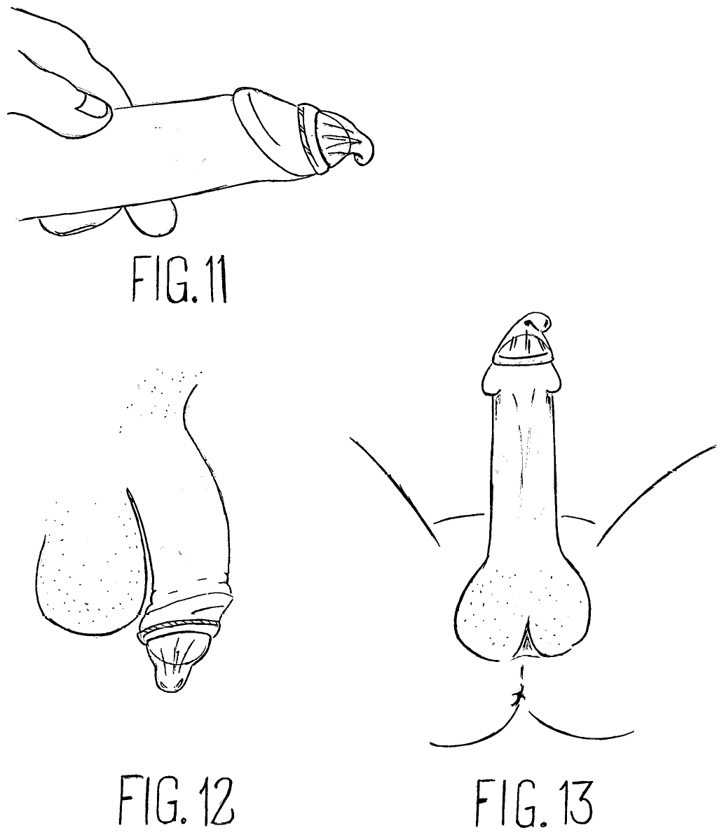

### DETAILED DESCRIPTION OF THE INVENTION

The invention termed Helmet, is comprised from preferred materials essentially derived from polymer's, rubber, plastic or anyone of the elastic compounds extracted from natural or synthetic substances. The most preferred material is Latex.

For the purposes of this description, and all specifications herein. A hypothetical comparison is necessary. Hypothetically, the concept of said helmet could be compared to grasping an ordinary condom, and cutting off the top portion of said condom at a length of about 4-centimeters. Once you have the top portion removed (which is just enough to cover the very tip of the male penis-head), this is the hypothetical concept of design for said helmet.

Thus, said helmet although one complete unit; is divided into separate elements in order to explain it functionality. First, said helmet has a vertical length comprising from the edge of an element termed Lip; to another element connected to it termed Reservior of about 5-centimeters; with the elasticity and pliability to expand further or decrease. Secondly, between the area's of said reservior and the helmet's midsection is an area termed Tank.

Starting from the top portion of said reservior, is a measurement of it's circumference of about ½-centimeter to 1½-centimeters; and said circumference expands even wider the closer it near's the top of said tank; gradually going from said ½-centimeter and 1½-centimeters, to about 3½-centimeters to 5-centimeters, with the elasticity and pliability to expand or decrease. In the hypothetical, said shape between the reservior and said tank has the appearance of the nipple on a baby-bottle. Where the nipple is wider in the midsection and then not only narrows as you travel up-wards, but also elongates.

Starting from the top of the tank to the edge of said lip; the circumference enlarges. Where the top of said tank peeked at 5-centimeters, it will now expand even further in circumference as it travel towards the lip to about 9-centimeters, with the same elasticity and pliability to expand or decrease. In respects to the reservior and said tank. The functionality of both these elements is as follows. The reservior is an encapsulated-dome-shape that catches fluids that spew forth from the urethra-tube located at the very tip of the penis-head. While the element termed the tank, has a function of assisting reservior in retaining excess fluids.

Due to the helmet's similar appearance to a baby-nipple and-or the top of a condom cut-off. It's a wonder how such an apparatus could be stabilized and fasten to the very tip of a penis-head, herein termed Crown. Well, on the opposite side of said lip, is an interior portion of said helmet and an element termed Belly-of-Lip. Along the total circumference of the belly-of-lip, will be an element attached to the surface termed an Adhesive-strip.

The adhesive-strip will comprise of gelantinous substances composed of any natural or synthetic substances; and act as an adhesive. In order to preserve the adhesive-strip's bonding integrity; an applicating element termed Wax-Coated-Pealable-Paper attaches to said adhesive-strip. The wan coated pealable-paper can be compared to the similar wax-or-plastic-coated-paper used to preserve the sticky adhesive on the back-side of a Postal Stamp or Band-aid.

### Usage of Helmet

The usage of said helmet is as simple as flipping the lip up-wards exposing said belly-of-lip and the wax-coated-pealable-paper protecting said adhesive-strip and removing said wax-coated-pealable-paper; then lining-up the tip of the penis-head with the entrance of the helmet, making sure the belly-of-lip, which supports said adhesive-strip, will make contact with said crown; then flipping the belly-of-lip down so the adhesive-strip bonds to the penis-head's skin. The adhesive-strip will bond to the total circumference of the penis-head in said crown area; creating a air-tight seal to keep fluids trapped within the helmet; while establishing a barrier that will not allow foreign fluids, viruses or bacteria's to enter within this isolated area of the helmet.

### Reason for Invention

A vast number of societies Male and Female population complain about the desensitizing affects of prophylactic's, especially condoms. In response to those individuals who champion this reason or excuse to forgo condoms. Only seems to place a negative stigma within our social fabric. The truth is, that traditional condoms (whether male or female condoms), due in fact, reduce sexual sinsation and stimulation in regards to the male and female genitalia.

Most condoms have a full rubbery coating that engulf's the entire length of the male penis; this is in regards to the female condom as well, called a Diaphragm.

Because the helmet occupies such a small area of real-estate on the penis. The helmet allows for full sinsation during sexual intercourse, while providing a measure of protection. The fact that traditional male and female condoms add a greater level of protection is of no consequence for the helmet. The truth is, due to many other reason's in addition to the few just stated; a greater number of men and women refuse to use condoms during sexual intercourse. Such a dangerous practice is a severe health hazard, not only to those participants, but to society as a whole. The spread of Sexually Transmitted Diseases and Infections (STD's or STI's), compounded with unwanted pregnancies; Makes this reality that much more detrimental to our society and the human race as a whole.

Now for the sake of this reasoning, consider the following factors. First, male condoms are sometimes difficult to apply to the penis. Often, a condom require's the user to place their penis in the condom, while giving the condom some slack for when the applicant rolls-down the remaining portion to the base of said penis. In situations like this, air-pockets are created within the condom requiring the applicant to re-start the application process all over again, fustrating the user and his partner; Second, there's a problematic issue with the size of condoms. Either the condom is to small or to big; Third, just before actual intercourse, application of a male condom requires the penis to be erect, which tends to interrupt the natural sexual process. Usually, the participants participate in a hot-and-heavy fore-play and then intercourse. But, with male or even female condoms for that matter; the scene goes from, hot-and-heavy to pause-and-wait, then apply (correctly), then back to hot-and-heavy and then actual sex. It is a well established opinion, that, condoms fustrate the natural process; Fourth, male condoms are prone to tear during intercourse, and some individuals will not stop during the sexual escapade to remove the torn condom and replace it with a new one, mainly because it's an interruption to the love-making. But, the helmet will alleviate those problems.

The helmet is designed to cover up just a slight portion of the penis. So there's less potential for tears. Also, 90-to-95% of the sexual sensation to the penis is restored; add that with the fact that said helmet is also roughly about 90-to-98% more compact and uses less material to make, means the helmet can help reduce waste. Another important fact, because of the helmet's unique element's, it allows for the user to apply said helmet with a flaccid penis. This attribute allows couples to engage in the natural process without any interruptions.

One of the strongest points to mention, is said helmet's design eliminates any and all excuses conjured-up by men and women to forgo any prophylactic measures or protection. This is a win, win for our society as a whole. By stripping individual's of their excuses to forgo even the most minimal amount of prophylactic protection. The helmet can reduce by leap's and bound's a heavy portion of the gap between STD's and unwanted pregnancies.

It cannot be ignored that the helmet leaves a lot of the length of the male penis exposed. So with the common knowledge of STI's spread through skin to skin contact, said helmet doesn't provide as much protection as the inventor would like to establish with such an idea. Nonetheless, it should be heavily considered, that, some protection is better then absolutely no protection. The helmet's design can still save live's and make an impact on society for the better. Even if that is only a small amount, every life matters.

The claimed invention is:

1. A prophylatic apparatus termed Helmet, designed to provide maximum sensation and stimulation for male genitalia during sexual intercourse, that comprises:
(a) A Latex-shell; one solid unit, designed to contain fluids expelled from male penis within sealed and encapsulated area; while preventing foreign fluids and bacteria's from entering sealed area of said latex-shell;
(b) A Reservior; which is an encapsulated-dome of said helmet and adjacent to;
(c) A Tank; which is the midsection of said helmet; and
(d) A Lip; which is at the edge of said helmet's opening; and
(e) A Belly-of-Lip; adjacent to said lip, and is the interior portion of said helmet; and
(f) An Adhesive-strip; attached to the area of said belly-of-lip; and
(g) A Wax-Coated-Pealable-Paper; attached to said adhesive-strip.

2. A helmet according to claim 1 with a circumference in-between the elements of said tank and said lip of about 9-centimeters with the elasticity and pliability to expand further or decrease; and comprising from preferred materials essentially made of polymer's, rubber's, plastic's or any elastic compounds derived from natural or synthetic materials; most preferred is latex.

3. A reservior according to claim 1 with a circumference of about ½-centimeters to 1½-centimeters and an elasticity and pliability to expand or decrease; and the reservior being an encapsulated dome shape, expanding from a narrow structure to a wider structure adjacent to the element said tank.

4. A tank according to claim 3 which extending down-ward, and connects to opening at end of the lip element; with a circumference of about 9-centimeters with an elasticity and pliability to expand or decrease.

5. A lip according to claim 4 and extending up-wards and connects to midsection element, said tank.

6. A belly-of-lip according to claim 1 located in the interior portion of said helmet, and providing support to said element adhesive-strip, attached to the interior portion of the belly-of-lip.

7. An adhesive-strip according to claim 6 and attached to element said belly-of-lip, in order to support said helmet's stablization and sealment.

8. An adhesive-strip according to claim 7; comprised of a gelantinous substance composed of any natural or synthetic substances used as a gluey and gummy adhesive, attached to interior portion of element, said belly-of-lip; dispersed evenly along total circumference of said belly-of-lip.

9. A wax-coated-pealable-paper according to claim 1, that attaches to said adhesive-strip element and spread out evenly among the total circumference of said adhesive-strip in order to protect said adhesive-strip's adhesive integrity.
